(19) United States
(12) Patent Application Publication
(10) Pub. No.: US 2018/0326132 A1
(43) Pub. Date: Nov. 15, 2018

(54) PUMP FOR TREATING CONGESTIVE HEART FAILURE

(71) Applicant: Edwards Lifesciences Corporation, Irvine, CA (US)

(72) Inventors: David Maimon, Haifa (IL); Gil Senesh, Mitzpe-Adi (IL)

(21) Appl. No.: 15/974,911

(22) Filed: May 9, 2018

Related U.S. Application Data
(60) Provisional application No. 62/505,298, filed on May 12, 2017.

(51) Int. Cl. A61M 1/10; A61M 1/12

(52) U.S. Cl. CPC A61M 1/1036; A61M 1/122; A61M 2205/3334; A61M 1/1086; A61M 1/106

(57) ABSTRACT

Disclosed herein are systems, devices and methods for heart assist pumps that are implanted using minimally-invasive techniques and that operate by generating a dynamic magnetic field outside of the body to cause a rotor of the heart assist pump to rotate and enhance blood flow through the pump. The disclosed systems, methods, and devices can be utilized in conjunction with a minimally-invasive operation to anchor a micro-pump device at a targeted location within the heart. The pump can be wholly located within the heart without any wires or cannulas penetrating the heart or the body of the patient. Use of this pump as a ventricular assist device can advantageously reduce recovery time, reduce complications, and potentially provide a solution for non-operable patients.

### CROSS-REFERENCE TO RELATED APPLICATIONS

The present application claims the benefit of U.S. Provisional Application No. 62/505,298, filed May 12, 2017, the entire disclosure of which is hereby incorporated herein by reference.

### BACKGROUND

### Field

The present disclosure generally relates to pumps for treating congestive heart failure, and, in particular, to implantable pumps that enhance blood flow through the heart.

### Description of Related Art

Congestive heart failure (CHF) is a disorder in which the heart fails to pump blood adequately to other organs in the body. This can result in a shortness of breath, fatigue and fluid retention (edema) and if left unchecked can lead to death within a few years. CHF is a progressive condition that may cause the heart muscle to weaken or stiffen over time leading to a reduction in cardiac output and may exacerbate symptoms of heart failure. This reduced cardiac output may cause a fall in arterial pressure leading to the activation of several compensatory reflexes. The sympathetic nervous system is stimulated, resulting in a direct increase in the force of contraction of the heart and a greater venous return as a response to venoconstriction. Long-term compensation includes the activation of the renin angiotensin system (RAS) and subsequent renal fluid retention. The combined effect of these responses can lead to the formation of edema, especially in the legs and ankles. If heart failure occurs in the left side of the heart, pulmonary edema can result which manifests as breathlessness. In advanced CHF, the severity of the symptoms can be disabling and often leads to hospitalization. An added consideration is sudden cardiac death, which can occur at any time during the course of CHF.

The failing heart is a result of a number of factors combining to reduce the efficiency of the heart as a pump. The most common dysfunction is an impairment of left ventricular function. As the blood flow from the heart slows, the blood returning to the heart through the veins can back-up, resulting in congestion in the tissues. This can lead to swelling in the legs and ankle and fluid retention in the lungs, which interferes with breathing and contributes to the characteristic shortness of breath seen in people with CHF.

### SUMMARY

In a first aspect, the present disclosure relates to a pump system configured to enhance blood flow in a heart. The system includes a pump unit having a support assembly with a frame that is radially collapsible for delivery in a catheter and expandable for deployment in an aorta of a patient. The pump unit also includes a rotor including a plurality of magnetic blades that are radially collapsible, the rotor being disposed within the frame and coupled to the support assembly. The system includes a wearable electromagnetic device configured to generate a dynamic magnetic field. The magnetic blades of the rotor rotate in the presence of the dynamic magnetic field.

In some embodiments of the first aspect, the pump unit is implanted in a heart of a patient and the wearable electromagnetic device is worn on an exterior of a body of the patient. In some embodiments of the first aspect, the support assembly further includes a plurality of support beams affixed to the frame. In a further embodiment, wherein the support assembly further includes a crossbar, the crossbar attached to at least one of the plurality of support beams at each end of the crossbar. In yet another further embodiment, the support assembly further includes a central shaft attached to the crossbar. In yet another further embodiment, the rotor further includes a propeller hub coupled to the crossbar, the propeller hub configured to support the plurality of magnetic blades and to allow the plurality of magnetic blades to rotate around the central shaft of the support assembly. In some embodiments of the first aspect, the pump unit is crimped and enclosed in a capsule.

In some embodiments of the first aspect, the pump unit does not include electrical wires electrically coupling the wearable electromagnetic device to the pump unit. In a further embodiment, the pump unit does not include any wires or cables that penetrate a wall of the heart. In some embodiments of the first aspect, the pump unit does not include electrical components configured to receive electrical power from a power source.

In some embodiments of the first aspect, in use, the pump unit is positioned within the heart to enhance blood flow through the heart. In a further embodiment, the pump unit does not include a cannula that penetrates a wall of the heart.

In a second aspect, a pump device is provided that is configured to enhance blood flow in a heart. The device includes a support assembly that is radially collapsible for delivery in a catheter and expandable for deployment in an aorta of a patient. The support assembly includes an expandable stent, the expandable stent comprising an interior surface and an exterior surface; a plurality of support beams coupled to an interior surface of the expandable stent; a crossbar attached to two of the plurality of support beams at ends of the crossbar; and a central shaft coupled to the crossbar. The device also includes a rotor that is radially collapsible for delivery in a catheter and expandable for deployment in an aorta of a patient. The rotor includes a propeller hub coupled to the central shaft so that the propeller hub is configured to rotate around a rotation axis parallel to the central shaft; and a plurality of magnetic blades attached to the propeller hub, each magnetic blade angled with respect to the rotation axis so that, in use, rotation of the propeller hub causes the plurality of magnetic blades to move and exert an axial force on fluid within the pump device.

In some embodiments of the second aspect, the support assembly at least partially includes a shape memory alloy configured to expand after implantation in the heart to secure the device at a targeted location. In some embodiments of the second aspect, the expandable stent further includes grabbing mechanisms that, in use, engage with tissue in the heart. In some embodiments of the second aspect, the expandable stent comprises a shape memory alloy. In a further embodiment, the shape memory alloy comprises Nitinol.

In some embodiments of the second aspect, the support assembly is configured to bend or fold at attachment points between the crossbar, the two of the plurality of support beams at the ends of the crossbar, and the central shaft. In a further embodiment, the rotor is configured to bend or fold at attachment points between the propeller hub and the plurality of magnetic blades. In a further embodiment, in a collapsed position, components of the support assembly and the rotor are bent or folded at attachment points between the components.

### BRIEF DESCRIPTON OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example of a collapsible pump unit for use in a heart to assist in pumping blood through the heart.

FIG. 2 illustrates another example pump unit having a support assembly with a single crossbar.

FIG. 3 illustrates another example pump unit having a support assembly with offset proximal and distal crossbars.

FIGS. 4A and 4B illustrate a cross-sectional view of a heart having a heart assist pump unit according to one or more embodiments implanted therein.

FIG. 5 illustrates the pump unit of FIG. 4 in use to assist in pumping blood through the aorta.

FIG. 6 illustrates an example wearable electromagnetic strap configured to generate a dynamic magnetic field.

FIG. 7 illustrates the wearable electromagnetic strap of FIG. 6 as worn by a patient.

FIG. 8 illustrates an example of a patient wearing an electromagnetic device that generates a dynamic magnetic field to drive a rotor of an implanted heart assist pump.

FIGS. 9A and 9B illustrate an example pump unit in a collapsed position (FIG. 9A) and in an expanded or deployed position (FIG. 9B).

FIG. 10 illustrates an example of a pump unit in a crimped or collapsed position to be inserted into a sheath or capsule.

FIGS. 11A and 11B illustrate two kits each having a wearable electromagnetic device and a pump unit.

FIG. 12 illustrates a flow chart of an example method of increasing blood flow through a heart using an implantable pump unit.

FIG. 13 illustrates a flow chart of an example method of implanting a heart assist pump within a heart of a patient.

FIG. 14 illustrates a flow chart of an example method 1400 of preparing a pump unit for implantation in a heart of a patient.

### DETAILED DESCRIPTION

The headings provided herein are for convenience only and do not necessarily affect the scope or meaning of any of the claimed embodiments.

### Overview

In humans and other vertebrate animals, the heart generally comprises a muscular organ having four pumping chambers, wherein the flow thereof is at least partially controlled by contraction of the walls of the heart. This contraction causes blood to flow through the circulatory system. If the heart muscle is incapable of pumping blood efficiently, blood flow may decrease to dangerous levels causing congestive heart failure (CHF). This may happen where the heart is weakened and/or the heart walls stiffen.

Most common practices to treat congenital heart failure (CHF) patients are based on using drugs to manipulate blood pressure and vessel contraction. Additional solutions involve the use of pumping units, such as so-called “VADs” (ventricular assist devices), that can be implanted to assist a functioning heart that does not have adequate pumping capability. Typical VADs are implanted during an open heart surgery. A left VAD (LVAD) takes blood from a lower chamber of the heart and help pump it to the aorta, just as a healthy heart would. The LVAD attaches to the patient's natural heart, and to a natural artery, and can be removed if the natural heart recovers. An implantable VAD may include a pump located inside of the body, with an associated power source located outside of the body. A cable may connect the pump to the power source through a small hole in the abdomen; the power source may have to be consistently operated. Some VADs are surgically implanted into the patient's abdominal cavity, while others remain outside the body and are placed in fluid communication with the heart via elongated cannulas.

Several types of surgically implantable pumps have been developed in an effort to provide a mechanical device for augmenting or replacing the blood pumping action of damaged or diseased hearts. Some of these pumps are designed to support single ventricular function. Such pumps usually support the left ventricle, which pumps blood to the entire body except the lungs, since it becomes diseased far more commonly than the right ventricle, which pumps blood only to the lungs. Other devices have been tested and used for providing biventricular function.

Disclosed herein are systems, devices and methods for heart assist pumps that are implanted using minimally-invasive techniques and that operate by generating a dynamic magnetic field outside of the body to cause a rotor of the heart assist pump to rotate and enhance blood flow through the pump. The disclosed systems, methods, and devices can be utilized in conjunction with a minimally-invasive operation to anchor a micro-pump device at a targeted location within the heart. This can advantageously reduce recovery time, reduce complications, and potentially provide a solution for non-operable patients.

The systems disclosed herein provide a mechanical device for augmenting the blood pumping action of damaged or diseased hearts. The systems include a pump unit implanted in the heart and an electromagnetic unit worn outside the body, wherein the electromagnetic unit generates a magnetic field that drives a rotor of the pump unit to enhance blood flow through the pump unit. This pump unit is a micro-pump device that can be placed inside the aorta in a minimally invasive procedure, such as a trans-femoral, a trans-epical or a trans-septical approach. The pump can be a part of the aortic valve, or a separate device located in the aorta, and may be configured to enhance flow during systole and to improve cardiac output. The pump system can be configured to apply a constant or pulsatile flow from the left ventricle to the aorta for a patient with CHF.

The micro-pump structure may comprise a self-expanding material, such as Nitinol wire or other shape memory alloy, such that once positioned with a delivery system device, the capsule may be dissolved or otherwise removed and the micro-pump may be self-extended to its actual desired structure. The pump unit includes two main parts that are physically attached: a frame and a rotor.

The frame may be similar to a Transcutaneous Aortic Valve Implantation (TAVI) frame. The frame can be a cylindrical wire frame. The frame is configured to anchor the micro-pump with sufficient radial strength to maintain the micro-pump during operation at a targeted location (e.g., at the aortic root or pulmonic root). The rotor component includes a propeller having tilted magnetic blades, the propeller being attached to the frame with a central pin that also serves as the rotor central spindle. In the presence of a magnetic field generated by an external device, the rotor rotates pushing the blood through the pump.

The pump unit is designed to be crimped and stored in a low-profile capsule until released. Following pump deployment, the micro-pump can be driven by an external wearable electromagnet that generates a dynamic magnetic field to control the movement of the rotor.

### Examples of Implantable Heart Assist Pumps

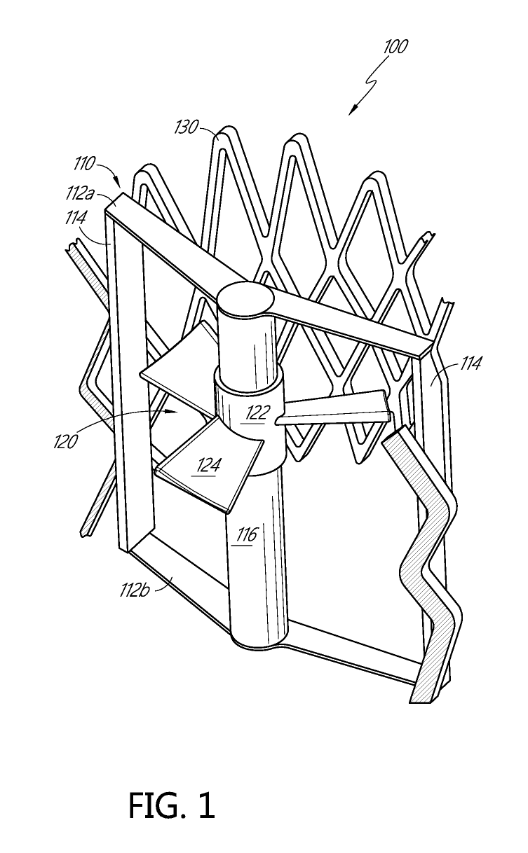

FIG. 1 illustrates an example of a collapsible pump unit 100 for use in a heart to assist in pumping blood through the heart. The pump unit 100 includes a rotor 120 and a support assembly 110 having an expandable frame or stent 130. When exposed to a changing magnetic field, the rotor 120 spins to increase fluid flow through the pump unit 100. For example, a separate device can be used to generate a changing magnetic field to cause the rotor 120 to spin, where the separate device is not physically coupled to the pump unit 100 and may be located outside of the heart and/or the body of a patient. The support assembly 110 and rotor 120 are configured to be collapsed and stored in a low-profile sheath or capsule for delivery to a targeted area, after which the support assembly 110 and rotor 120 expand for implantation and operation.

The rotor 120 is mounted on the support assembly 110 that includes the expandable frame 130, such as a stent, that is adapted to be positioned at a target location within the body duct (e.g., the aorta). The expandable frame 130 forms a conduit through which fluids flow. The expandable frame 130 can be configured to deploy the pump unit 100 by the use of suitable deployment mechanisms, such as a catheter or other similar devices. In some embodiments, the pump unit 100 is configured for percutaneous positioning and deployment. In such embodiments, the expandable frame 130 is configured to be posed in two positions, a crimped position where the cross-section of the conduit is small so as to permit advancing the pump unit 100 towards its target location, and a deployed position where the expandable frame 130 is extended radially by forces exerted from within (e.g., by a deployment mechanism) or self-expanded (e.g., due to the use of shape memory alloys) to provide support against the body duct wall, to secure the pump unit 100 in position, and to open itself so as to allow fluid flow through the conduit. The expandable frame 130 can be annular or it may be provided in other shapes that relate to the cross-section shape of the targeted location within a patient.

The support assembly 110 is configured to provide elements that support the rotor 120 so that it can spin in the presence of a suitable electromagnetic field. The support assembly 110 includes a distal crossbar 112a and a proximal crossbar 112b (e.g., proximal and distal with respect to a direction of a force exerted on fluids by the pump unit 100), with each crossbar 112a, 112b mechanically coupled to support beams 114 and a central shaft 116. The crossbars 112a, 112b are mechanically coupled to the central shaft 116 at or near the ends of the central shaft 116. Additionally, the central shaft 116 attaches at or near a midpoint of each crossbar 112a, 112b. The crossbars 112a, 112b are mechanically coupled to the support beams 114 at or near the ends of the support beams 114. Additionally, the support beams 114 attach at or near the ends of each crossbar 112a, 112b. In this way, the central shaft 116 is supported by the crossbars 112a, 112b and the support beams 114.

The rotor 120 is mechanically coupled to the central shaft 116 in a way that allows the rotor 120 to spin. The rotor 120 includes a propeller hub 122 with a plurality of blades 124 extending from the propeller hub 122. The blades 124 can include material that is permanently magnetic. In some embodiments, the blades 124 are at least partially made of a material that is a permanent magnet, such as ceramic magnets, ferrite magnets, alnico magnets, injection molded magnets, flexible magnets, or the like. In various embodiments, the blades 124 can be at least partially coated with a magnetic material or coating. In certain embodiments, the blades 124 include one or more permanent magnets attached to individual blades 124. Examples of permanent magnets include, without limitation, rare earth magnets such as neodymium magnets or samarium-cobalt magnets. These permanent magnets and other components of the pump unit 100 can be coated with suitable biocompatible coatings to reduce or prevent rejection of the pump unit 100. Due to the magnetic properties of the blades 124, the rotor 120 can be caused to rotate around the central shaft 116 when exposed to a tailored dynamic electromagnetic field. Rotation of the blades 124 generates an axial force on a fluid (e.g., blood) that is present within the pump unit 100. Thus, the pump unit 100 can assist in pumping blood.

The pump unit 100 operates by generating a magnetic with tailored properties (e.g., magnetic field strength and direction as a function of position and/or time) to cause a force to be exerted on the blades 124 through interaction of the magnetic field of the magnetic blades 124 and the tailored magnetic field. As described in greater detail herein, the magnetic field can be generated by a device that is wholly separate from the pump unit 100.

The fit between the propeller hub 122 and the central shaft 116 can be configured so that the rotor 120 principally rotates along a single, desired axis of rotation, reducing or preventing wobbling of the rotor 120. In some embodiments, the rotor 120 includes bearings that allow the propeller hub 122 to fit securely on the central shaft 116 while allowing the propeller hub 122 and blades 124 to rotate. In such embodiments, the bearings may be secured to the central shaft 116 to inhibit or prevent the propeller hub 122 from travelling longitudinally along the central shaft 116.

In certain embodiments, an inner radius of the propeller hub 122 is slightly larger than the outer radius of the central shaft 116. In such embodiments, the fluid within the heart can fill the space between the central shaft 116 and the propeller hub 122 to provide lubrication between these components allowing the rotor 120 to rotate when secured to the central shaft 116. In various embodiments, the central shaft 116 includes an indented portion with an outer radius that is smaller than the rest of the central shaft 116, wherein the propeller hub 122 is positioned itself within the indented portion to inhibit or prevent the propeller hub 122 from travelling longitudinally along the central shaft 116.

The support assembly 110 is also configured to position and secure the pump unit 100 in a targeted or desired location within the heart so that during operation the pump unit 100 remains substantially fixed in place. The support beams 114 are mechanically coupled to the expandable frame 130 to support the crossbars 112a, 112b and central shaft 116 so that they remain substantially fixed when the pump unit 100 is implanted in the heart of a patient. The force of the expandable frame 130 against the walls of the heart can be sufficient to reduce or prevent movement of the pump unit 100 when the rotor 120 rotates, causing an increase of blood flow through the pump unit.

The expandable frame 130 can be configured to change size (e.g., collapse and expand) to allow the pump unit 100 to be implanted in a heart and to secure the pump unit 100 within the heart once implanted. The expandable frame 130 can be made from plastically-expandable materials, shape memory alloys such as nickel titanium (nickel titanium shape memory alloys, or NiTi, as marketed, for example, under the brand name Nitinol), or other biocompatible metals. The percutaneously implantable pump unit 100 with the expandable frame 130 can be suitable for crimping into a narrow configuration for positioning and expandable to a wider, deployed configuration so as to anchor in position in the targeted location.

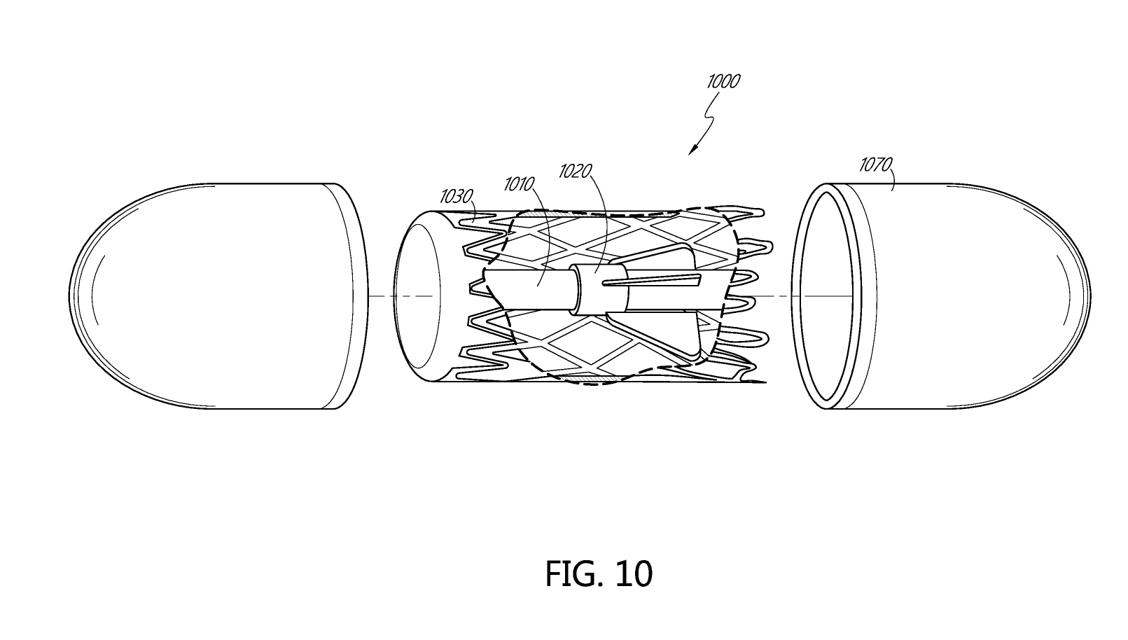

In certain implementations, the expandable frame 130 can include plastically-expandable materials that permit crimping of the pump unit 100 to a smaller profile for delivery and expansion of the pump unit 100 using a deployment device. In various implementations, the expandable frame 130 can include self-expanding material such as a shape memory alloy. This self-expanding pump unit 100 can be crimped to a smaller profile and held in the crimped state with a restraining device such as a sheath or capsule (e.g., as described in greater detail herein with respect to FIG. 10). When the pump unit 100 is positioned at or near the target site, the restraining device is removed, dissolved, or destroyed to allow the pump unit 100 to self-expand to its expanded, functional size. For example, collapsible pump units 100 can be crimped to a compressed state and percutaneously introduced in the compressed state using a catheter and expanded to a functional size at the targeted position by use of an expander on the catheter or by utilization of a self-expanding frame or stent 130.

The expandable frame 130 made of a shape memory alloy can cause the support assembly 110 to have a deployed diameter when the support assembly is not acted on by any external force so that the expandable frame 130 contacts the walls of the targeted location with sufficient force to secure the pump unit in place. In certain embodiments, the expandable frame 130 includes one or more projections (e.g., hooks, barbs, or anchors) to penetrate the native tissue at the targeted location to further secure the pump unit 100 in place.

In some embodiments, the support assembly 110 is constructed with materials so that it can be radially compressed into a compressed state for delivery through the patient's vasculature, and can self-expand to a natural, uncompressed or functional state having a preset or targeted diameter. Thus, the support assembly 110 expands or tends toward a targeted diameter when free of external forces. In certain implementations, the support assembly 110 can be expanded beyond its targeted diameter to an over-expanded diameter (e.g., using a deployment mechanism). After the support assembly 110 is in this over-expanded state, the support assembly 110 returns toward its targeted diameter (or naturally recoils to the targeted diameter).

As illustrated, the expandable frame 130 can be a net-like frame. This configuration can be adapted to crimp evenly so as to present a narrow configuration for deployment and can extend to occupy the passage at the target location for implantation in a body duct. However, it is to be understood that other configurations that provide similar or equivalent functionality may be used for the expandable frame 130. Additional examples and details of the support assembly 110 and the expandable frame 130 are provided in U.S. Pat. Nos. 6,730,118, 6,893,460, and 8,460,366, the entire contents of each of which is hereby incorporated herein by reference for all purposes.

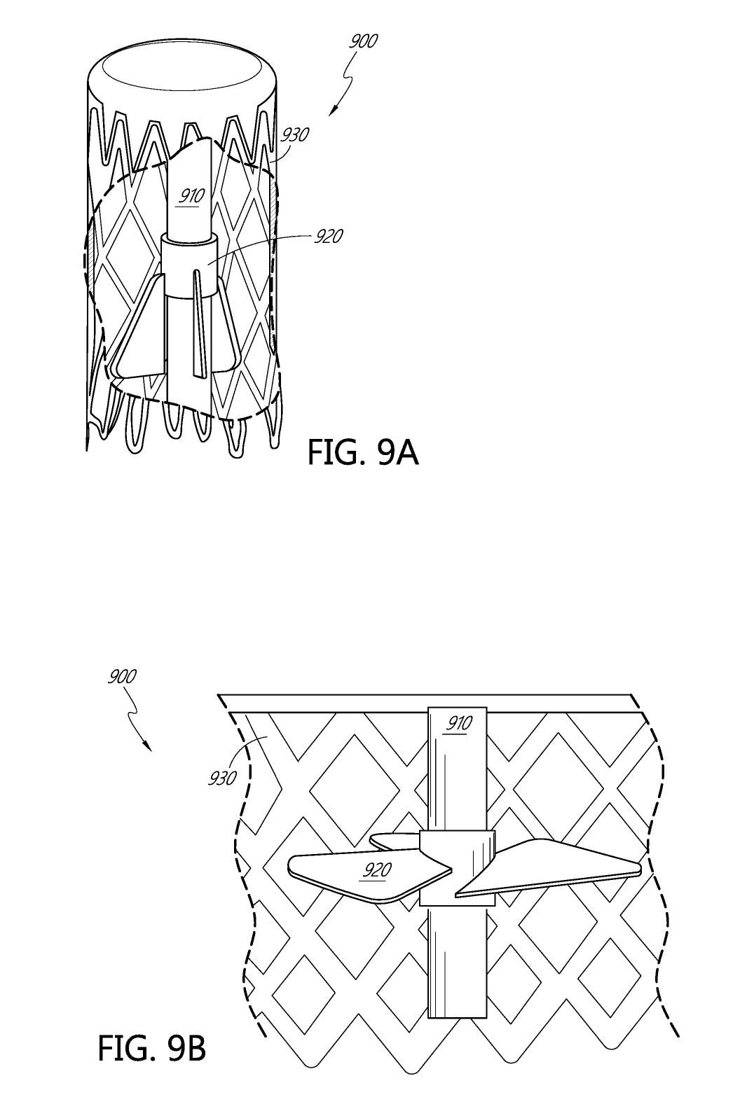

As described in greater detail herein with respect to FIGS. 9A and 9B, the support assembly 110 and rotor 120 can be configured to collapse when the expandable frame 130 is in a crimped position (e.g., during deployment) and to expand when the expandable frame 130 is in a deployed position (e.g., during operation of the pump unit 100). Accordingly, where the crossbars 112a, 112b attach to the support beams 114 and where the crossbars 112a, 112b attach to the central shaft 116 can be made to bend or fold so that the elements remain attached to one another while still remaining within the conduit formed by the expandable frame 130. The attachment of the support beams 114 to the expandable frame 130 can be accomplished in several ways. For example, the support beams 114 can be attached or affixed to the expandable frame by sewing each support beam 114 to several anchoring points on the expandable frame 130. Other attachment mechanisms include, for example and without limitation, riveting, pinning, adhering, welding, casting or molding the support beams 114 on the expandable frame 130, or any other suitable way of attachment.

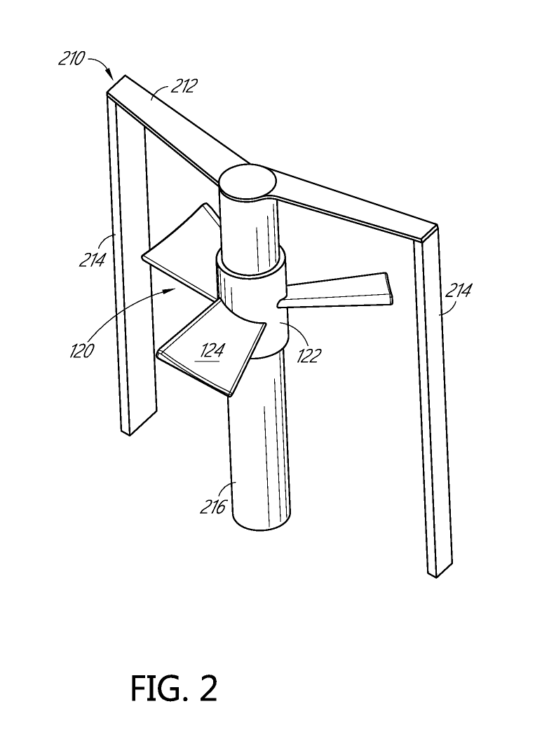

FIG. 2 illustrates an example pump unit, similar to the pump unit 100 of FIG. 1, with a different support assembly 210. It should be noted that the expandable frame 130 has been removed from the illustration for clarity sake. However, it is to be understood that the support assembly 210 includes an expandable frame or stent similar to the expandable frame 130 described herein with reference to FIG. 1. The support assembly 210 includes a single crossbar 212 attached to a central shaft 216 and support beams 214. The support beams 214 attach at or near a distal end of the crossbar 212. The support beams 214 are also attached to the expandable frame as described elsewhere herein. The central shaft 216 attaches to the crossbar 212 at or near a midpoint of the crossbar 212.

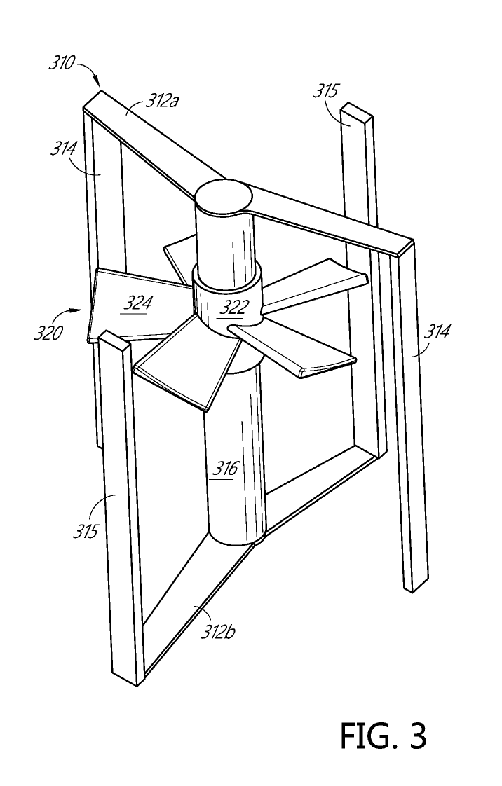

FIG. 3 illustrates another example pump unit, similar to the pump unit 100 of FIG. 1, with a different support assembly 310 and a different rotor 320. It should be noted that the expandable frame 130 has been removed from the illustration for clarity sake. However, it is to be understood that the support assembly 310 includes an expandable frame or stent similar to the expandable frame 130 described herein with reference to FIG. 1. The support assembly 310 includes a distal crossbar 312a attached to a central shaft 316 and support beams 314. The support beams 314 attach at or near the ends of the distal crossbar 312a. The support beams 314 are also attached to the expandable frame as described elsewhere herein. The central shaft 316 attaches to the distal crossbar 312a at or near a midpoint of the distal crossbar 312a. Similarly, the support assembly 310 includes a proximal crossbar 312b attached to the central shaft 316 and support beams 315. The support beams 315 attach at or near the ends of the proximal crossbar 312b. The support beams 315 are also attached to the expandable frame as described elsewhere herein. The central shaft 316 attaches to the proximal crossbar 312b at or near a midpoint of the proximal crossbar 312b.

The distal and proximal crossbars 312a, 312b are rotated relative to one another. As illustrated, the distal and proximal crossbars 312a, 312b are perpendicular to one another, but other configurations and variations on the relative orientation of the crossbars falls within the scope of this disclosure. Due at least in part to their relative orientations, the support assembly 310 includes two pairs of support beams 314, 315 attached respectively to distal and proximal crossbars 312a, 312b.

Other configurations are possible as well. For example, the support assembly can include a plurality of crossbars attached to a distal (or proximal) end of the central shaft, with the crossbars being rotated relative to one another (e.g., the contemplated rotation being around the longitudinal axis formed by the central shaft), similar to spokes of a wheel. In such configurations, support beams can extend longitudinally from the ends of each crossbar. These crossbar “spokes” can be on the distal or proximal end of the central shaft.

In some implementations, a plurality of corresponding proximal and distal crossbars can be included in the support assembly (FIG. 1 illustrates a single pair of corresponding distal and proximal crossbars). In such implementations, support beams can extend between the ends of corresponding crossbars (e.g., as illustrated in FIG. 1 for a single pair of corresponding distal and proximal crossbars).

In certain implementations, a crossbar can extend from the central shaft to a support beam attached to the expandable frame rather than from one side of the expandable frame to an opposite side of the expandable frame. As an illustrative example, three such crossbars can be included in the support assembly where the crossbars are rotated 120 degrees relative to one another around the longitudinal axis of the central shaft. This concept can be extended for any suitable number of crossbars extending from the central shaft to a support beam attached to the expandable frame.

The rotor 320 includes a propeller hub 322 having five blades 324. The rotor 320 can include any suitable number of blades 324. For example, the rotor 320 can include at least 2 blades, at least 3 blades, at least 4 blades, at least 5 blades, at least 7 blades, at least 10 blades, etc. Similarly, the rotor 320 can include less than or equal to 10 blades, less than or equal to 8 blades, less than or equal to 6 blades, less than or equal to 5 blades, less than or equal to 4 blades, less than or equal to 3 blades, etc.

### Implantation and Operation of Heart Assist Pump Units

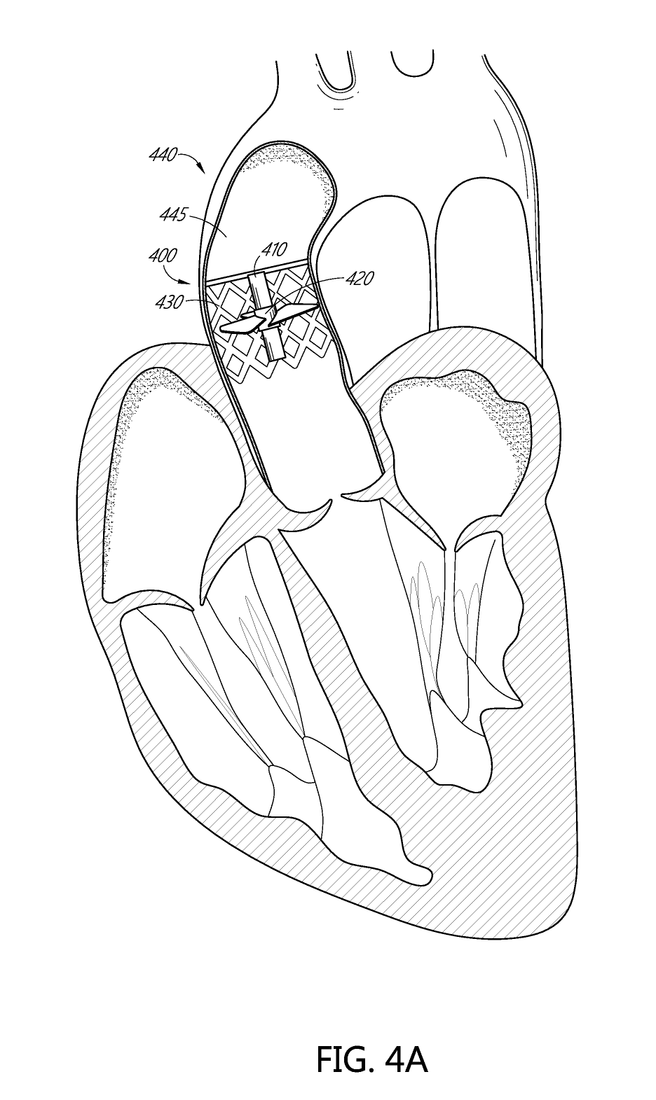

FIG. 4A illustrates a cross-sectional view of a heart 440 having a heart assist pump unit 400 according to one or more embodiments implanted therein. The pump unit 400 includes a rotor 420 and a support assembly 410 having an expandable stent 430, the pump unit 400 configured to increase the flow of blood through the pump unit 400 or to assist in pumping blood through the aorta 445. The expandable stent 430 is configured to expand so that at least a portion of the expandable stent 430 contacts the walls of the aorta 445 to secure the pump unit 400 in place. Although illustrated as part of the aortic valve 445, it is to be understood that the pump unit 400 can be implanted in any portion of the heart (e.g., aortic root, pulmonic root, arteries, etc.) to assist in pumping blood into, out of, and/or through the heart 440. For example, FIG. 4B illustrates a cross-sectional view of the heart 440 having the heart assist pump unit 400 implanted in a pulmonary artery 447.

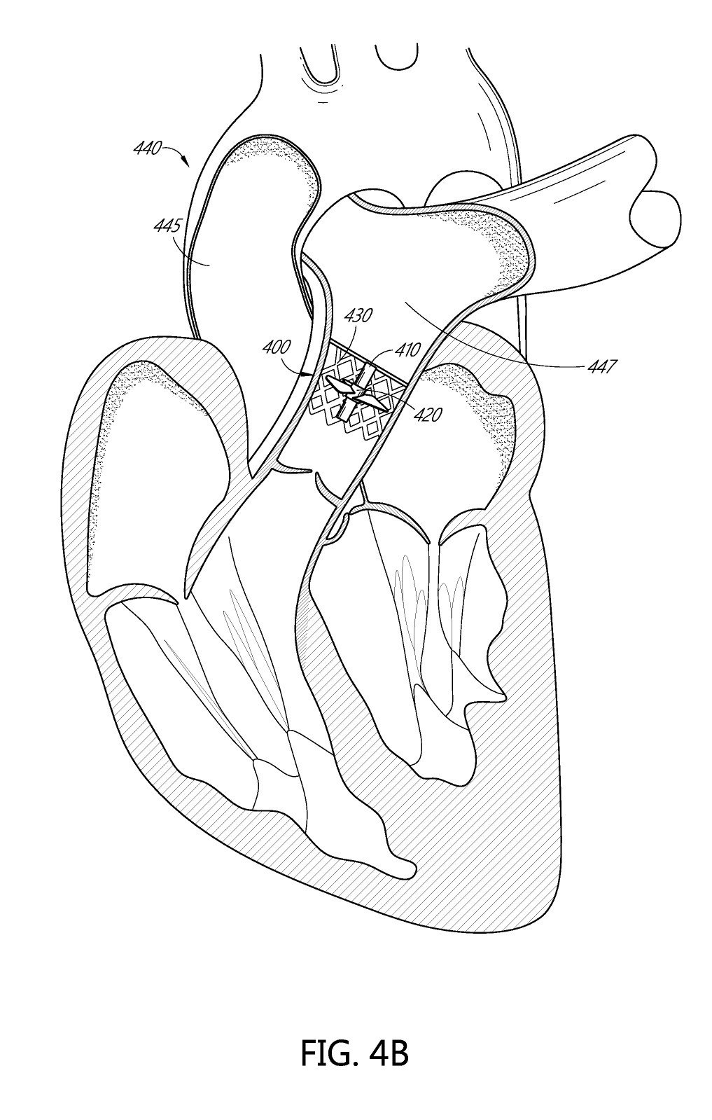

FIG. 5 illustrates the pump unit 400 of FIG. 4A in use to assist in pumping blood through the aorta 445. In use, the rotor 420 is caused to rotate due to a dynamic electromagnetic field generated near the pump unit 400. As the rotor 420 turns, supported by the support assembly 410 and held in place in the aorta 445 by the expandable stent 430, an axial force is applied to the fluid in the aorta 445 to increase the flow 450 of the fluid through the pump unit 400. The pump unit 400 is configured to enhance flow during systole and to improve cardiac output. In some embodiments, the pump unit 400 is configured to apply a constant flow or a pulsatile flow from the left the ventricle to the aorta for a patient with CHF. The pump unit 400 can be implanted in the aorta 445 or other blood vessel in a minimally-invasive procedure.

Advantageously, the pump unit 400 functions in response to electromagnetic fields generated using a device (or devices) that is physically separate from the pump unit 400. For example, such a device may be located outside of the heart 440 and/or outside of the patient's body. The device can be configured to generate a tailored, dynamic electromagnetic field that induces mechanical rotation of magnetic blades of the pump unit 400. In certain implementations, the pump unit 400 operates without any electrical power being generated inside of the body. For example, all of the electrical power used to drive the rotor 420 by way of generating a dynamic magnetic field is provided using a system that is external to body. Thus, the pump unit 400 does not include any component that receives electrical power to generate a magnetic field to drive the rotor 420, as in other electromagnetic pumps. This beneficially allows for implantation and operation of the pump unit 400 without the use of any electrical power source, such as a battery, implanted in the heart 440 or body of the patient.

Advantageously, the pump unit 400 does not include any electrical components. The pump unit 400 does not receive electrical power through wires or other conductive elements. The pump unit 400 does not receive electrical power to cause the rotor 420 to rotate. Accordingly, the pump unit 400 can be a mechanical implant, as opposed to an electrical or electromechanical implant. Consequently, in some embodiments, the pump unit 400 can be implanted and operated with no electrical components inside the heart 440 or body. Moreover, in some embodiments, the pump unit 400 can be implanted and operated without wires or cables penetrating a patient's tissue or organs such as the heart or skin. Beneficially, the pump unit 400 may be safer to implant and operate than other ventricular assist devices that utilize electrical components implanted in the heart and/or body. As described herein, the pump unit 400 is powered (e.g., the rotor 420 is caused to rotate) by application of a dynamic electromagnetic field generated outside of the heart 440 (or body). This differs from devices that are powered through electrical induction, or the wireless transfer of electrical power between electrical components. For example, the pump unit 400 does not receive electrical power that is converted into mechanical energy through electrical or electromechanical components. Rather, the mechanical rotation of the rotor 420 is merely a natural consequence of the interaction between the magnetic blades of the rotor 420 and the dynamic electromagnetic field.

Advantageously, the pump unit 400 is wholly contained within the aorta 445 when in use. Thus, no conduits or electrical leads penetrate through the walls of the heart 440 to operate the pump unit 440 (e.g., to cause the rotor 420 to rotate or to assist in pumping blood through the aorta 445). Similarly, the pump unit 440 can be operated by using a device to generate a dynamic electromagnetic field, where the device is entirely located outside of the heart 440 and/or outside of a patient. When the device is located outside of the heart 440, the pump unit 400 can be operated without a direct electrical connection to the device generating the magnetic field. Accordingly, the pump unit 400 can be implanted and operated using minimally invasive procedures. For example, the micro-pump 400 can be implanted inside the aorta 445 in a minimally invasive procedure, such as a trans-femoral, a trans-epical, or a trans-septical approach. Recovery times may be improved due at least in part to the pump unit 400 being operated without any penetrating wires.

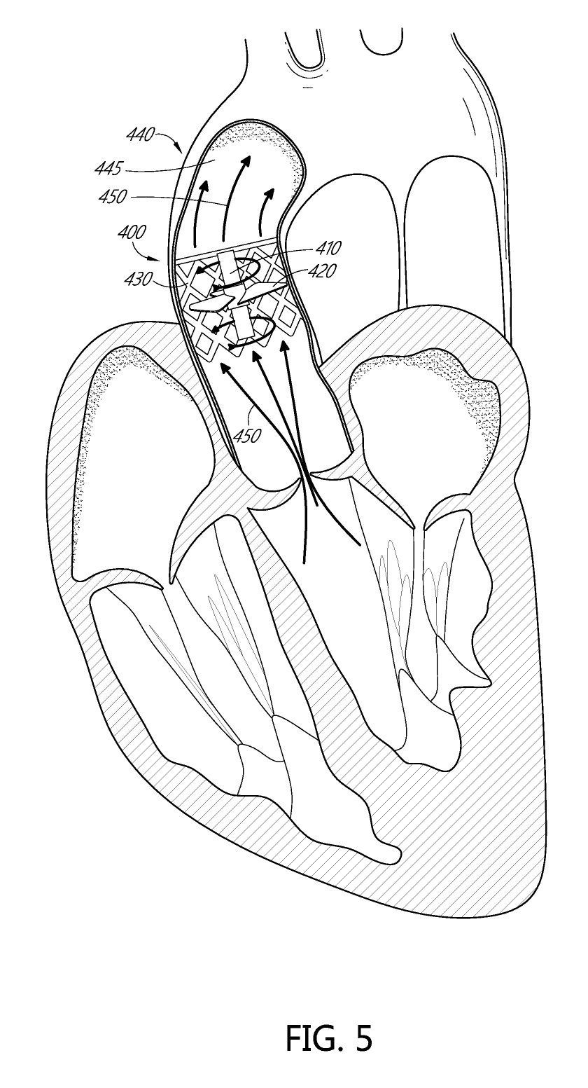

FIG. 6 illustrates an example wearable electromagnetic strap 660 configured to generate a dynamic magnetic field 662. The strap 660 is an example of a device that can be used to generate the magnetic field that drives the rotor of the heart assist pump devices described herein. The strap 660 can be configured to generate the magnetic field 662 by driving a tailored electrical current through conductors within the strap 660. These conductors can be configured to be wrapped around a core so that when an electrical current passes through the conductors, a magnetic field is generated. The strap 660 can be coupled to a suitable power source (not shown) that includes a controller configured to control current through the strap 660 to generate the targeted electromagnetic field 662.

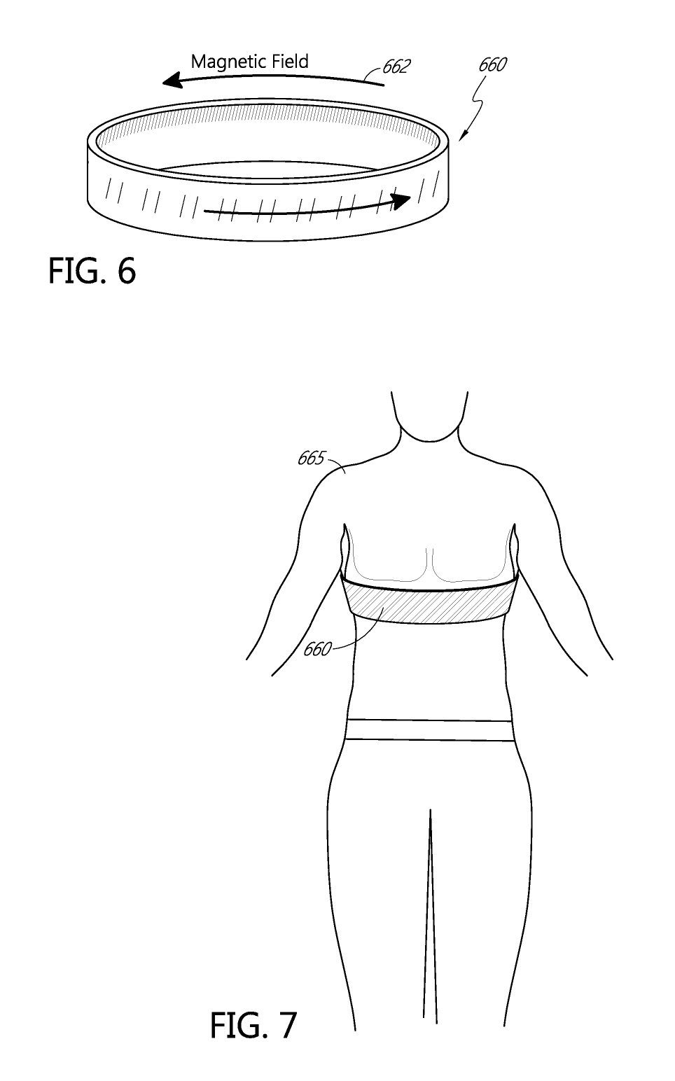

FIG. 7 illustrates the wearable electromagnetic strap 660 of FIG. 6 as worn by a patient 665. In this configuration, the strap 660 can be configured to induce mechanical rotation of a rotor of heart assist pump devices described herein. The strap 660 can be worn around the torso, chest, or abdomen of the patient 665 when operating such pump devices.

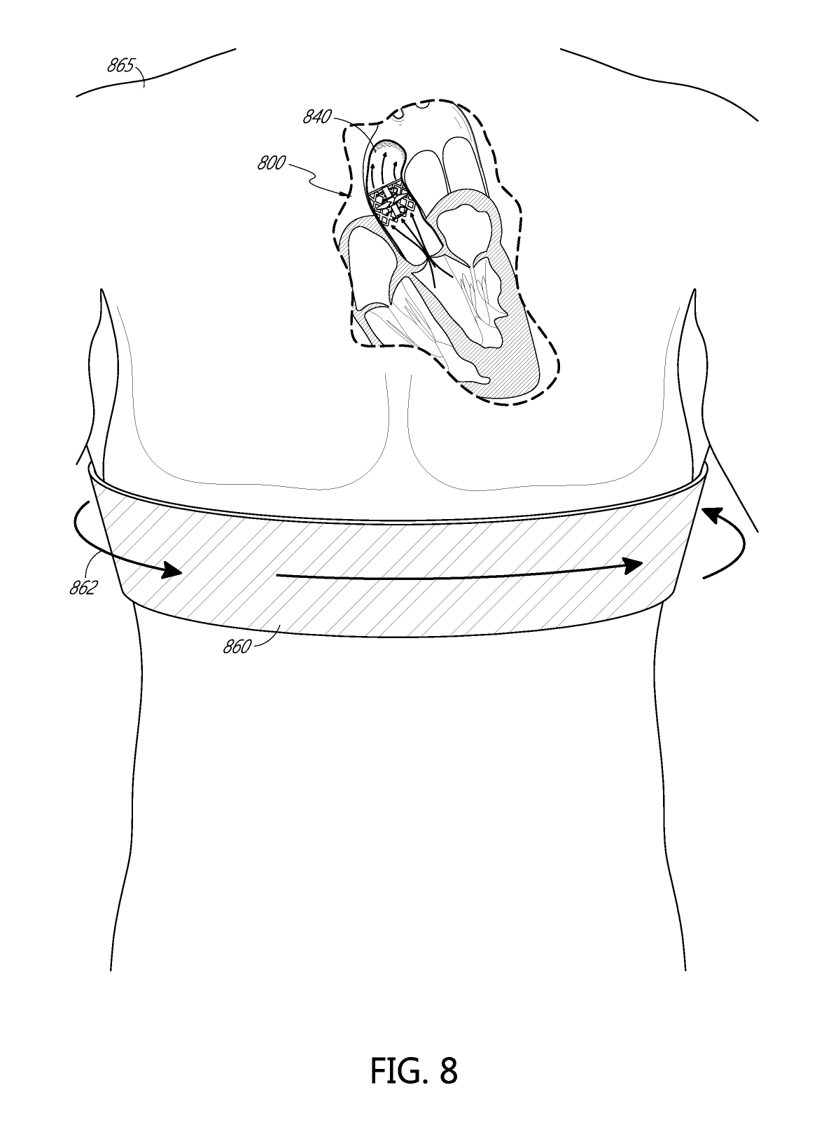

FIG. 8 illustrates an example of a patient 865 wearing an electromagnetic device 860 that generates a dynamic magnetic field 862. The generated magnetic field 862 causes an implanted pump unit 800 to assist in pumping blood through the aorta 840 of the patient 865. A power source (not shown) may be electrically coupled to the electromagnetic device 860 to cause a tailored current to flow through the electromagnetic device 860 thereby generating a desired or targeted dynamic electromagnetic field 862 to cause the pump unit 800 to increase blood flow through the aorta 840. The strap 860 can be configured to generate a targeted magnetic field at the location of the pump unit 800 to drive the rotor of the pump unit at a targeted rotational rate. The strap 860 can be an external, wearable band-like electromagnet for wirelessly powering and controlling the pump unit 800.

Advantageously, the pump unit 800 is powered without the use of wires passing from the strap 860 to the pump unit. Accordingly, the pump unit 800 is contained within the heart 840 without wires penetrating the walls of the heart 840. Advantageously, the pump unit 800 is configured to enhance blood flow without the use of cannulas or conduits that direct blood out of and back into the heart 840. Accordingly, the pump unit 800 is contained within the heart 840 without cannulas or conduits penetrating the walls of the heart 840. The pump unit 800 does not include conductive coils inside the body to generate the tailored electromagnetic field that drives the rotor of the pump unit 800. Furthermore, the power source that ultimately provides the electrical power that drives the pump unit 800 (via generated magnetic fields) is located outside of the heart 840 and/or body. This allows the pump unit 800 to be implanted and operated without any incisions through the walls of the heart 840.

In some embodiments, the pump unit 800 differs from pumps that operate using transcutaneous energy transfer due at least in part to the pump unit 800 being driven by a magnetic field that is generated by a device that is outside of the body. For comparison, certain pumps that operate by way of transcutaneous energy transfer involve wirelessly transferring electrical power into the body and/or heart where that electrical power is used to drive an electrical current through coils in a stator to generate a magnetic field that drives a rotor, both the stator and rotor being located inside of the body. Similarly, certain other pumps that operate by way of transcutaneous energy transfer involve wirelessly transferring electrical power into the body and/or heart where that electrical power is converted into mechanical energy using electromechanical components that are located inside of the body. In contrast, the pump unit 800 does not include a stator located inside of the heart 840 and/or body. Similarly, the pump unit 800 does not include electromechanical components that are configured to convert electrical voltage and/or current into mechanical motion. Thus, as used herein, the rotor of the pump unit 800 is not considered an electromechanical component because it is the interaction between the magnetic blades of the rotor and the magnetic fields generated by external devices that results in mechanical motion rather than the direct conversion of electrical power to mechanical motion.

In some embodiments, the strap 860 is configured to receive feedback related to the blood flow through the heart 840. The strap 860 can be configured to adjust operating parameters to increase or decrease the amount of assistance provided by the pump unit 800 based at least in part on the received feedback.

In some embodiments, the strap 860 includes one or more user interface features that receive input and/or display information. These user interface features may be used to monitor performance of the pump unit 800. These user interface features may also be used to manually adjust properties of the generated magnetic field 862. In certain embodiments, the strap 860 can be configured to be controlled by an electronic device, such as a remote control, computer, smartphone, tablet, or the like. For example, a smartphone can be associated with the strap 860 (e.g., through a wired or wireless connection) and an application on the smartphone can be used to monitor performance of the strap 860, to monitor performance of the pump unit 800, to monitor parameters associated with blood flow, to adjust properties of the strap 860 (e.g., electrical current through the strap 660), and the like.

FIGS. 9A and 9B illustrate an example pump unit 900 in a collapsed position (FIG. 9A) and in an expanded or deployed position (FIG. 9B). The pump unit 900 includes a support assembly 910 and a rotor 920 that are both collapsible. The support assembly 910 includes a support stent 930 that is also collapsible. The support assembly 910 and the rotor 920 are made with a deployable construction that is adapted to be initially crimped in a narrow configuration suitable for catheterization through the body duct to a target location. In some embodiments, the support assembly 910 and the rotor 920 are adapted to be deployed by exerting substantially radial forces from within by means of a deployment device to a deployed state in the target location. In some embodiments, the support assembly 910 and the rotor 920 are adapted to be deployed by way of self-expanding materials that expand the pump unit 900 from the collapsed state to the deployed state in the target location.

The components of the support assembly 910 can be made to bend and/or fold at desired or targeted locations within the support assembly 910. For example, where crossbars attach to support beams and/or a central shaft, the crossbars can be made to bend and/or fold at or near the attachment points. Similarly, the components of the rotor 920 can be made to bend and/or fold at desired or targeted locations. For example, where magnetic blades attach to a propeller hub, the blades can be made to bend and/or fold at or near these attachment points. The support stent 930 can be made to expand and collapse due at least in part to the ductility of the material used in the stent 930 and/or the mesh or net-like construction of the stent 930. The support assembly 910 includes support beams of fixed length attached to the stent 930, as described in greater detail herein with reference to FIGS. 1-3, and these support beams can remain attached to the stent 930 in the collapsed and expanded states.

FIG. 10 illustrates an example of a pump unit 1000 in a crimped or collapsed position to be inserted into a sheath or capsule 1070. The pump unit 1000 is shown with an expandable frame 1030 that is partially cut away to reveal the support structure 1010 and rotor 1020 in the crimped position. The pump unit can be designed to be crimped and stored in a low-profile capsule 1070 until released. Upon release, the pump unit 1000 can be expanded and/or can self-expand to a deployed position at a targeted location. The capsule 1070 can be configured to dissolve, self-destruct, be removed, or otherwise open so that the pump unit can expand at the targeted location.

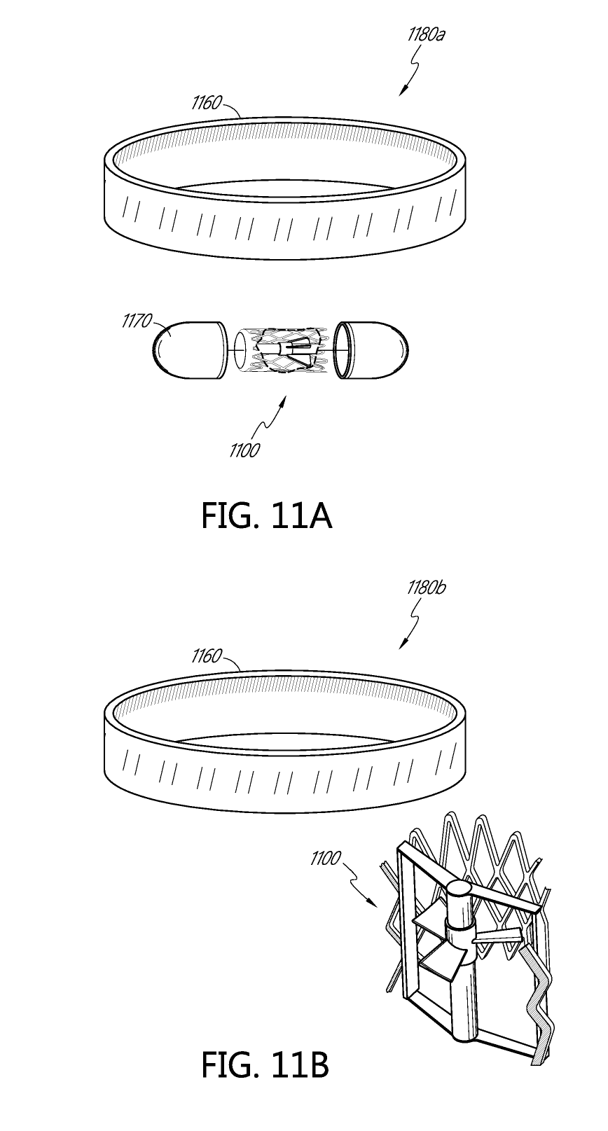

FIGS. 11A and 11B illustrate two kits 1180a, 1180b each having a wearable electromagnetic device 1160 and a pump unit 1100. In the kit 1180a, the pump unit 1100 is provided in a crimped position and may be provided within a sheath or capsule 1170 suitable for implantation, examples of which are described in greater detail with reference to FIG. 10. In the kit 1180b, the pump unit 1100 is provided in an expanded or deployed state. The pump unit 1100 may be crimped in preparation for implantation using a variety of suitable implantation and deployment mechanisms. In some embodiments, the kits 1180a, 1180b can include the device 11160, the capsule 1170 and/or pump unit 1100 along with a delivery catheter (not shown) and/or specialized tool (not shown) for securing the capsule 1170 during implantation, opening the capsule 1170, and removing the pump unit 1100 from the capsule 1170.

### Methods of Operating, Implanting, and Preparing Heart Assist Pumps

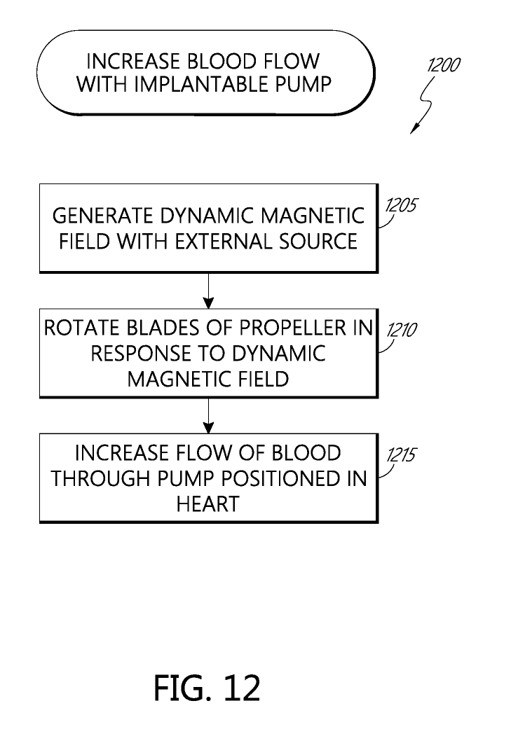

FIG. 12 illustrates a flow chart of an example method 1200 of increasing blood flow through a heart using an implantable pump unit. The pump unit can be any of suitable embodiment of the various pump units described herein, wherein the pump unit includes a support assembly and a rotor. The method 1200 describes the use of the pump unit implanted in the heart, wherein the pump unit is not coupled to a power source outside of the heart and the pump unit does not include any electrical components in the heart that are configured to drive the rotor of the pump unit.

In block 1205, a dynamic magnetic field is generated using a device that is external to the heart and/or body of the patient. Examples of suitable devices are described in greater detail herein with respect to FIGS. 6-8. In some embodiments, the device is configured to adjust the properties of the generated magnetic field in response to measured, sensed, or provided feedback. In some embodiments, the properties of the generated magnetic field can be manually adjusted.

In block 1210, the rotor of the pump unit rotates in response to the generated magnetic field. The rotor includes magnetic blades coupled to a propeller hub. In the presence of the generated magnetic field, the magnetic blades experience a force that translates into rotational motion. The dynamic magnetic field can be tailored to induce a targeted rate of rotation of the rotor. The pump unit does not include any electrical components within the heart that are configured to generate a magnetic field to cause the rotor to rotate.

In block 1215, rotation of the rotor causes an axial force to be applied to fluid through the pump unit. This axial force enhances the flow of blood through the pump unit, thereby increasing the flow of blood through the heart.

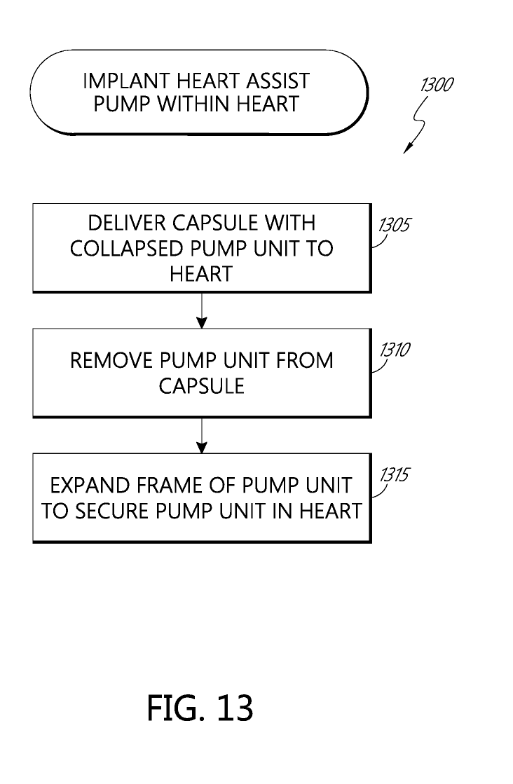

FIG. 13 illustrates a flow chart of an example method 1300 of implanting a heart assist pump within a heart of a patient. The heart assist pump can be an embodiment of any of the pump units described herein. The heart assist pump includes a support assembly and a rotor coupled to the support assembly. The support assembly includes an expandable stent, support beams coupled to the expandable stent, at least one crossbar coupled to the support beams, and a central shaft coupled to the at least one crossbar. The rotor is coupled to the central shaft in such a way that allows rotation of the rotor around the central shaft.

The heart assist pump is configured to be powered by way of a magnetic field generated outside of the heart. Thus, the heart assist pump does not include any electrical wires that couple to devices or systems outside of the heart. The heart assist pump is configured to enhance blood flow within the heart. Thus, the heart assist pump does not include any cannulas that direct blood flow out of and back into the heart. Consequently, there are no wires, cables, or cannulas that penetrate the walls of the heart. This allows the heart assist pump to be implanted using a catheter or other similar minimally invasive procedure. This may also allow the pump to be implanted without incisions in the heart. Advantageously, this may reduce recovery times after implantation of the heart assist pump, reduce complications associated with the implantation of the pump, and may potentially be a solution for patients that may otherwise be non-operable.

In block 1305, a capsule is delivered to the heart, wherein the capsule contains a heart assist pump in a collapsed position, examples of which are described herein with respect to FIGS. 9A and 10. The capsule can be a low-profile sheath configured to be delivered through an artery to the heart. The capsule can be attached to a catheter or other similar device. In some embodiments, the catheter includes a specialized tool that is configured to secure the capsule in place during delivery to the heart. In certain embodiments, the capsule can be delivered to the heart without incisions to the heart and without stopping the heart or putting the heart on bypass.

In block 1310, the pump unit is removed from the capsule. This may be accomplished by destroying the capsule (e.g., by dissolving the capsule or breaking it) or by opening the capsule. In some embodiments, the tool used to secure and deliver the capsule can be used to open the capsule as well. This tool may be configured to open the capsule and/or expel the pump unit from inside the capsule.

In block 1315, the support assembly of the pump unit expands so that the frame or support stent contacts at least a portion of the interior of the heart to secure the pump in place. In some embodiments, the pump includes materials, such as shape memory alloys, that cause the pump to self-expand to a predetermined diameter to secure the pump in place. In some embodiments, the tool used to secure the capsule during delivery and to remove the pump from the capsule can also be used to expand the pump. For example, the tool can include a mechanism that applies a radially outward force from within the pump unit to expand the rotor and the support assembly including the expandable frame to secure the frame in place in the heart. In some embodiments, the frame includes grabbing mechanisms that engage with the tissue in the heart to secure the pump in place.

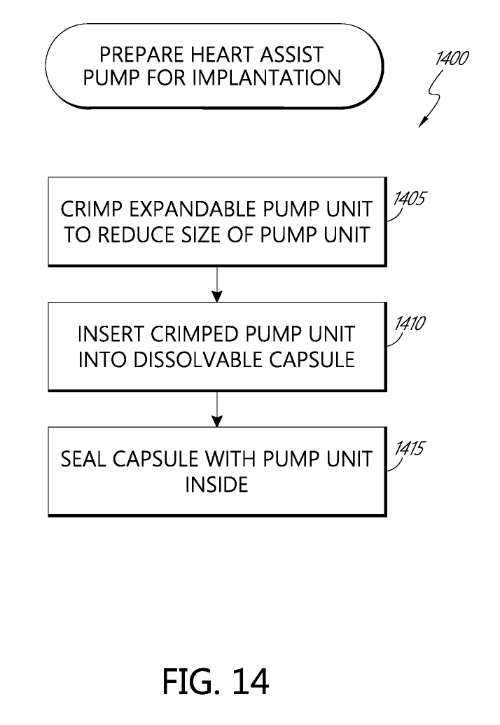

FIG. 14 illustrates a flow chart of an example method 1400 of preparing a heart assist pump for implantation in a heart of a patient. The heart assist pump can be an embodiment of any of the pump units described herein. The heart assist pump includes a support assembly and a rotor coupled to the support assembly. The support assembly includes an expandable stent, support beams coupled to the expandable stent, at least one crossbar coupled to the support beams, and a central shaft coupled to the at least one crossbar. The rotor is coupled to the central shaft in such a way that allows rotation of the rotor around the central shaft. The support assembly and the rotor are configured to be collapsible.

In block 1405, the pump unit is crimped to reduce the radial size of the pump. The pump unit can be crimped using any suitable manual or automatic crimping tool. The support assembly and rotor are configured to bend and/or fold in a particular way to allow crimping of the heart unit without permanently breaking or destroying the pump unit or its constituent parts.

In block 1410, the crimped pump unit is housed within a sheath, such as a dissolvable, destroyable, or openable capsule. The capsule can be a sterile package. In some embodiments, the capsule can be included with a delivery catheter and/or specialized tool for securing the capsule during implantation, opening the capsule, and removing the pump unit from the capsule.

In block 1415, the capsule is closed and/or sealed with the crimped pump unit inside. The pump unit in the capsule can be stored until it is needed for a procedure, at which point a physician can remove the capsule with the pump unit and then implant the pump unit in a patient.

### Additional Embodiments

As used herein, the terms “collapsible,” “expandable,” and other related words are used interchangeably to indicate that the disclosed structures can change their radial size to become smaller for delivery (e.g., a collapsed or crimped state) and to become larger for implantation and operation in the heart (e.g., an expanded or deployed state). It should be understood that decreasing the radial size of the structure may increase, for example, its longitudinal dimension. However, for the purposes of this disclosure, this is still considered to be collapsible.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described herein. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

Reference throughout this specification to “certain embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics can be combined in any suitable manner, as would be apparent to one of ordinary skill in the art from this disclosure, in one or more embodiments.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. A pump system configured to enhance blood flow in a heart, the system comprising:
a pump unit comprising:
a support assembly including a frame that is radially collapsible for delivery in a catheter and expandable for deployment in an aorta of a patient; and
a rotor including a plurality of magnetic blades that are radially collapsible, the rotor being disposed within the frame and coupled to the support assembly; and
a wearable electromagnetic device configured to generate a dynamic magnetic field,
wherein the magnetic blades of the rotor rotate in the presence of the dynamic magnetic field.

2. The system of claim 1, wherein the pump unit is implanted in a heart of a patient and the wearable electromagnetic device is worn on an exterior of a body of the patient.

3. The system of claim 1, wherein the support assembly further includes a plurality of support beams affixed to the frame.

4. The system of claim 3, wherein the support assembly further includes a crossbar, the crossbar attached to at least one of the plurality of support beams at each end of the crossbar.

5. The system of claim 4, wherein the support assembly further includes a central shaft attached to the crossbar.

6. The system of claim 5, wherein the rotor further includes a propeller hub coupled to the crossbar, the propeller hub configured to support the plurality of magnetic blades and to allow the plurality of magnetic blades to rotate around the central shaft of the support assembly.

7. The system of claim 1, wherein the pump unit is crimped and enclosed in a capsule.

8. The system of claim 1, wherein the pump unit does not include electrical wires electrically coupling the wearable electromagnetic device to the pump unit.

9. The system of claim 8, wherein the pump unit does not include any wires or cables that penetrate a wall of the heart.

10. The system of claim 1, wherein the pump unit does not include electrical components configured to receive electrical power from a power source.

11. The system of claim 1, wherein in use, the pump unit is positioned within the heart to enhance blood flow through the heart.

12. The system of claim 11, wherein the pump unit does not include a cannula that penetrates a wall of the heart.

13. A pump device configured to enhance blood flow in a heart, the device comprising:
a support assembly that is radially collapsible for delivery in a catheter and expandable for deployment in an aorta of a patient, the support assembly comprising:
an expandable stent, the expandable stent comprising an interior surface and an exterior surface;
a plurality of support beams coupled to an interior surface of the expandable stent;
a crossbar attached to two of the plurality of support beams at ends of the crossbar; and
a central shaft coupled to the crossbar; and
a rotor that is radially collapsible for delivery in a catheter and expandable for deployment in an aorta of a patient, the rotor comprising:
a propeller hub coupled to the central shaft so that the propeller hub is configured to rotate around a rotation axis parallel to the central shaft; and
a plurality of magnetic blades attached to the propeller hub, each magnetic blade angled with respect to the rotation axis so that, in use, rotation of the propeller hub causes the plurality of magnetic blades to move and exert an axial force on fluid within the pump device.

14. The device of claim 13, wherein the support assembly at least partially includes a shape memory alloy configured to expand after implantation in the heart to secure the device at a targeted location.

15. The device of claim 13, wherein the expandable stent further includes grabbing mechanisms that, in use, engage with tissue in the heart.

16. The device of claim 13, wherein the expandable stent comprises a shape memory alloy.

17. The device of claim 16, wherein the shape memory alloy comprises Nitinol.

18. The device of claim 13, wherein the support assembly is configured to bend or fold at attachment points between the crossbar, the two of the plurality of support beams at the ends of the crossbar, and the central shaft.

19. The device of claim 18, wherein the rotor is configured to bend or fold at attachment points between the propeller hub and the plurality of magnetic blades.

20. The device of claim 19, wherein, in a collapsed position, components of the support assembly and the rotor are bent or folded at attachment points between the components.
